(19) United States
(12) Patent Application Publication
(10) Pub. No.: US 2020/0306216 A1
(43) Pub. Date: Oct. 1, 2020

(54) CURE FOR ALZHEIMER'S DISEASE

(71) Applicant: Anis Ahmad, Rock Island, IL (US)

(72) Inventor: Anis Ahmad, Rock Island, IL (US)

(21) Appl. No.: 16/371,660

(22) Filed: Apr. 1, 2019

(51) Int. Cl. A61K 31/202; A61P 25/28; A61P 31/22; A61P 5/50; A61P 29/00; A61K 31/198; A61K 38/28; A61K 33/24; A61K 33/08; A61K 31/122; A61K 31/40; A61K 31/4704; C07K 16/40

(52) U.S. Cl. CPC A61K 31/202; A61P 25/28; A61P 31/22; A61P 5/50; A61P 29/00; A61K 31/198; C07K 16/40; A61K 33/24; A61K 33/08; A61K 31/122; A61K 31/40; A61K 31/4704; A61K 38/28

(57) ABSTRACT

The use of a combination therapy to treat Alzheimer's disease is disclosed herein. The disclosed combination therapy comprises administration of a therapeutically effective amount of a combination of one or more agents used for treatment of the herpes simplex virus, one or more agents used for reducing insulin resistance, one or more anti-inflammatory agents, one or more agents for treatment of atheromatous plaques, and one or more agents for treating impaired neurogenesis.

### BACKGROUND

### Field of the Invention

The present disclosure relates to the use of a combination therapy to treat Alzheimer's disease.

### Description of the Related Art

Alzheimer's disease is a chronic neurodegenerative disease that is the cause of 60-70% of cases of dementia. See, e.g., Burns, A., et al. “Alzheimer's Disease,” BMJ, 2009, 338, doi: 10.1136/bmj.b158; World Health Organization, “Dementia Fact Sheet,” 2017, available online at www.who.int/en/news-room/fact-sheets/detail/dementia. Alzheimer's disease was first described in 1906 by German psychiatrist and neuroanatomist Alois Alzheimer. Hippius, H., et al. “The Discovery of Alzheimer's Disease,” Dialogues Clin. Neurosci. 2003, 5(1), 101. There are approximately 30-35 million people worldwide with Alzheimer's disease. See World Health Organization, supra. Alzheimer's disease affects about 6% of people who are 65 years of age and older. Burns, A., et al., supra. Alzheimer's disease is one of the most financially costly diseases in developed countries. See, e.g., Bonin-Guillaume, S., et al. “The Economical Impact of Dementia,” Presse Medicate, 2005, 34, 35, doi: 10.1016/s0755-4982(05)83882-5.

Alzheimer's disease is frequently seen in patients also suffering from diabetes mellitus. See Benedict, C., et al. “Insulin Resistance as a Therapeutic Target in the Treatment of Alzheimer's Disease: A State-of-the-Art Review,” Front. Neurosci. 2018, 12, 215, doi: 10.3389/fnins.2018.00215. The fundamental abnormalities observed in Alzheimer's disease represent the effects of brain insulin resistance and deficiency. de la Monte, S. M. “Type 3 Diabetes is Sporadic Alzheimer's Disease: Mini-Review,” Eur. Neuropsychopharmacol. 2014, 24(12), 1954-60. The molecular and biochemical consequences of Alzheimer's disease also overlap with those of Type 1 and Type 2 diabetes mellitus. Id. Thus it has been suggested that Alzheimer's disease is properly termed Type 3 diabetes. See Id.

There is also a significant overlap between vascular dementia and Alzheimer's disease in terms of clinical expression, risk factors, and postmortem brain autopsy. See Ravona-Springer, R., et al. “Is the Distinction between Alzheimer's Disease and Vascular Dementia Possible and Relevant?” Dialogues Clin. Neurosci. 2003, 5(1), 7.

Alzheimer's disease results in damage to regions of the brain that control memory and cognitive functions. Patients ultimately lose their memory and ability to learn, reason, communicate, and carry out daily activities. Alzheimer's disease is characterized by the presence of three primary histopathological hallmarks: senile plaques that are extra cellular aggregates composed of amyloid β peptide (Aβ), neurofibrillary tangles (NFT) that are intracellular aggregates composed of hyperphosphorylated forms of tau proteins, and extensive loss of neurons from different areas of the brain. See Benedict, C., et al., supra.

There is no known cure for Alzheimer's disease. Alzheimer's disease has multiple etiological factors, and each factor must be treated for any possible treatment to be completely effective. The etiological factors for Alzheimer's disease are:
(1) infection of the brain with the herpes simplex virus, see Piacentini, R., et al. “HSV-1 and Alzheimer's: More than a Hypothesis,” Front. Pharmacol. 2014, 5, 97, doi: 10.3389/fphar.2014.00097;
(2) insulin resistance in the brain, see, e.g., Benedict, C., et al., supra; Escudero, C. A., et al. “Pro-Angiogenic Role of Insulin: From Physiology to Pathology,” Front. Physiol. 2017, 8, 204, doi: 10.3389/fphys.2017.00204;
(3) amyloid β deposits (Aβ) and neurofibrillary tangles (NFT) in the brain, see Benedict, C., et al., supra;
(4) atherosclerosis of the cerebrovascular region and amyloid angiopathy, see, e.g., Ghiso, J., et al. “Cerebral Amyloid Angiopathy and Alzheimer's Disease,” Hirosaki Igaku, 2010, 61(suppl.), S111-S124; Roher, A. E., et al. “Atherosclerosis of Cerebral Arteries in Alzheimer Disease,” Stroke, 2004, 35(suppl. I), 2623-27, doi: 10.1161/01.STR.0000143317.70478.b3;
(5) endothelial dysfunction of blood vessels and a lack of angiogenesis, see, e.g., Escudero, C. A., et al., supra;
(6) chronic inflammation of the brain, see, e.g., Akiyama H., et al. “Inflammation and Alzheimer's Disease,” Neurobiol. Aging, 2000, 21(3), 383-421; Fuster-Matanzo, A., et al. “Role of Neuroinflammation in Adult Neurogenesis and Alzheimer Disease: Therapeutic Approaches,” Mediators Inflamm. 2013, 2013, 260925, doi: 10.1155/2013/260925; and
(7) an extensive loss of neurons and impaired neurogenesis, see, e.g., Fujimaki, S., et al. “Diabetes and Stem Cell Function,” BioMed Res. Int. 2015, doi: 10.1155/2015/592915.
   Any treatment for Alzheimer's disease that does not treat all of these factors will be only partially effective at best.

Thus there remains a need for a treatment for Alzheimer's disease that addresses all the relevant etiological factors.

### SUMMARY

The use of a combination therapy to treat Alzheimer's disease is disclosed herein. The disclosed combination therapy comprises administration of a therapeutically effective amount of a combination of one or more agents used for treatment of the herpes simplex virus, one or more agents used for reducing insulin resistance, one or more anti-inflammatory agents, one or more agents for treatment of atheromatous plaques, and one or more agents for treating impaired neurogenesis.

In some embodiments, the disclosed combination therapy comprises the administration of a therapeutically effective amount of a combination of (1) lysine to treat the herpes simplex virus; (2) one or more agents selected from the group consisting of insulin, magnesium oxide, chromium picolinate, coenzyme Q10, carvedilol, and metformin for reducing insulin resistance; (3) one or more agents selected from the group consisting of a statin, eicosapentaenoic acid (EPA) or a derivative thereof, and montelukast for reducing inflammation; (4) one or more agents selected from the group consisting of insulin, magnesium oxide, chromium picolinate, coenzyme Q10, carvedilol, and metformin; one or more agents selected from the group consisting of a statin, eicosapentaenoic acid (EPA) or a derivative thereof, and montelukast; and one or more PCSK9 inhibitors for treatment of atheromatous plaques; and (5) one or more agents from the group consisting of paroxetine and EPA for treating impaired neurogenesis.

### DETAILED DESCRIPTION OF THE ILLUSTRATED EMBODIMENTS

The use of a combination therapy to treat Alzheimer's disease is disclosed herein. The disclosed combination therapy comprises administration of a therapeutically effective amount of a combination of one or more agents used for treatment of the herpes simplex virus, one or more agents used for reducing insulin resistance, one or more anti-inflammatory agents, one or more agents for treatment of atheromatous plaques, and one or more agents for treating impaired neurogenesis.

In some embodiments, the disclosed combination therapy comprises the administration of a therapeutically effective amount of a combination of (1) lysine to treat the herpes simplex virus; (2) one or more agents selected from the group consisting of insulin, magnesium oxide, chromium picolinate, coenzyme Q10, carvedilol, and metformin for reducing insulin resistance; (3) one or more agents selected from the group consisting of a statin, eicosapentaenoic acid (EPA) or a derivative thereof, and montelukast for reducing inflammation; (4) one or more agents selected from the group consisting of insulin, magnesium oxide, chromium picolinate, coenzyme Q10, carvedilol, and metformin; one or more agents selected from the group consisting of a statin, eicosapentaenoic acid (EPA) or a derivative thereof, and montelukast; and one or more PCSK9 inhibitors for treatment of atheromatous plaques; and (5) one or more agents from the group consisting of paroxetine and EPA for treating impaired neurogenesis.

### Proposed Mechanism of Action

The use of a combination of one or more agents used for treatment of the herpes simplex virus, one or more agents used for reducing insulin resistance, one or more anti-inflammatory agents, one or more agents for treatment of atheromatous plaques, and one or more agents for treating impaired neurogenesis treats all the major etiological factors of Alzheimer's disease. This leads to reversal of the secondary pathologies associated with Alzheimer's disease and results in curing of an affected patient.

Alzheimer's disease has multiple etiological factors, and each factor must be treated for any possible treatment to be completely effective. The etiological factors for Alzheimer's disease are:
(1) infection of the brain with the herpes simplex virus, see Piacentini, R., et al., supra;
(2) insulin resistance in the brain, see, e.g., Benedict, C., et al., supra; Escudero, C. A., et al., supra;
(3) amyloid β deposits (Aβ) and neurofibrillary tangles (NFT) in the brain, see Benedict, C., et al., supra;
(4) atherosclerosis of the cerebrovascular region and amyloid angiopathy, see, e.g., Ghiso, J., et al., supra; Roher, A. E., et al., supra;
(5) endothelial dysfunction of blood vessels and a lack of angiogenesis, see, e.g., Escudero, C. A., et al., supra;
(6) chronic inflammation of the brain, see, e.g., Akiyama H., et al., supra; Fuster-Matanzo, A., et al., supra; and
(7) an extensive loss of neurons and impaired neurogenesis, see, e.g., Fujimaki, S., et al., supra.
   The disclosed combination therapy treats Alzheimer's disease by treating all of the aforementioned etiological factors.

### Treatment of Herpes Simplex Virus

A therapeutically effective amount of one or more agents used for treatment of the herpes simplex virus is administered as part of the disclosed combination therapy. In some embodiments, a therapeutically effective amount of lysine is administered for treatment of the herpes simplex virus infection of the brain. Lysine is known to suppress viral replication of the herpes simplex virus and inhibit cytopathogenicity of the virus. See, e.g., Griffith, R. S., et al. “A Multicentered Study of Lysine Therapy in Herpes Simplex Infection,” Dermatologica, 1978, 156(5), 257-67.

### Reduction of Insulin Resistance

A therapeutically effective amount of one or more agents used for reducing insulin resistance is administered as part of the disclosed combination therapy. Reducing insulin resistance is an extremely important aspect of treatment, as it addresses multiple etiological factors of Alzheimer's disease, as discussed below.

Insulin resistance promotes the formation of amyloid β plaques (Aβ) and neurofibrillary tangles (NFT), both of which are major pathological hallmarks of Alzheimer's disease. See Benedict, C., et al., supra. Insulin receptors are widely distributed throughout the brain, with a high concentration in the olfactory bulb, hypothalamus, and hippocampus. Id. It is proposed herein that reducing insulin resistance in the brain leads to the removal of the amyloid β deposits (Aβ) and neurofibrillary tangles (NFT) that are characteristic of Alzheimer's disease.

Reducing insulin resistance also reduces amyloid angiopathy. See, e.g., Saito, S., et al. “New Therapeutic Approaches for Alzheimer's Disease and Cerebral Amyloid Angiopathy,” Front. Aging Neurosci. 2014, doi: 10.3389/fnagi.2014.00290. The amyloid deposits in and around cerebral blood vessels lead to changes in the integrity of the blood-brain barrier, see, e.g., Magaki, S., et al. “The Effects of Cerebral Amyloid Angiopathy on Integrity of the Blood-Brain Barrier,” Neurobiol. Aging, 2018, 70, 70-77, extravasation of plasma protein, see, e.g., Takechi, R., et al. “Chylomicron Amyloid-Beta in the Aetiology of Alzheimer's Disease,” Atheroscler. Suppl. 2008, 9(2), 19-25, doi: 10.1016/j.atherosclerosissup.2008.05.010, edema formation, see, e.g., Merlini, G., et al. “Amyloidosis: Pathogenesis and New Therapeutic Options,” J. Clin. Oncol. 2011, 29(14), 1924-33, doi: 10.1200/JCO.2010.32.2271, and the release of inflammatory mediators and matrix metalloproteases that cause partial degradation of basal lamina with partial hemorrhagic complications. See, e.g., Ghiso, J., et al., supra.

Endothelial dysfunction of blood vessels is also caused by insulin resistance. See, e.g., Janus, A., et al. “Insulin Resistance and Endothelial Dysfunction Constitute a Common Therapeutic Target in Cardiometabolic Disorders,” Mediators Inflamm. 2016, 2016, 3634948. Endothelial dysfunction results in a lack of angiogenesis. See, e.g., Bierhansl, L., et al. “Central Role of Metabolism in Endothelial Cell Function and Vascular Disease,” Physiology (Bethesda), 2017, 32(2), 126-40.

In some embodiments, a therapeutically effective amount of one or more agents from the group consisting of insulin, magnesium oxide, chromium picolinate, coenzyme Q10, carvedilol, and metformin is administered as part of the combination therapy. In some embodiments, a therapeutically effective amount of one or more agents from the group consisting of insulin, magnesium oxide, chromium picolinate, and coenzyme Q10 is administered as part of the combination therapy. In some embodiments, therapeutically effective amounts of insulin, magnesium oxide, chromium picolinate, and coenzyme Q10 are administered as part of the combination therapy.

Chromium picolinate reduces insulin resistance and exhibits anti-inflammatory properties. See, e.g., Balk, E. M., et al. “Effect of Chromium Supplementation on Glucose Metabolism and Lipids: A Systematic Review of Randomized Controlled Trials,” Diabetes Care, 2007, 30(8), 2154-2163, doi: 10.2337/dc06-0996.

Magnesium oxide reduces insulin resistance and exhibits anti-inflammatory properties. See, e.g., Rodriguez-Morán, M., et al. “Oral Magnesium Supplementation Improves Insulin Sensitivity and Metabolic Control in Type 2 Diabetic Subjects: A Randomized Double-Blind Controlled Trial,” Diabetes Care, 2003, 26(4), 1147-52; Moslehi, N., et al. “Effects of Oral Magnesium Supplementation on Inflammatory Markers in Middle-Aged Overweight Women,” J. Res. Med. Sci. 2012, 17(7), 607-14.

Coenzyme Q10 reduces insulin resistance and is an antioxidant. See, e.g., Mantle, D. “Coenzyme Q10 Supplementation for Diabetes and Its Complications: An Overview,” Br. J. Diabetes, 2017, 17, 145-48.

Carvedilol reduces insulin resistance and stimulates insulin receptors. See, e.g., U.S. Pat. No. 8,492,426; Muniz, P., et al. “Antiplatelet Activity of β-blockers: New Light on Existing Data,” Br. J. Clin. Pharmacol. 2014, 78, 937-39; Bakris, G. L., et al. “Metabolic Effect of Carvedilol vs. Metoprolol with Hypertension,” J. Am. Med. Assoc. 2004, 18, 2227-36. Carvedilol also exhibits anti-inflammatory properties. See, e.g., Yuan, Z., et al. “Cardioprotective Effects of Carvedilol on Acute Autoimmune Myocarditis: Anti-Inflammatory Effects Associated with Antioxidant Property,” Am. J. Physiol. Heart Circ. Physiol. 2004, 286, 83-90.

Metformin reduces insulin resistance and also has anti-inflammatory properties. See Saisho, Y. “Metformin and Inflammation: Its Potential Beyond Glucose-Lowering Effect,” Endocr. Metab. Immune Disord. Drug Targets, 2015, 15, 196-205.

### Treatment of Atherosclerosis in Cerebrovascular Region

Atherosclerosis of the cerebrovascular region may be treated by removing atheromatous plaques therein. Thus, a therapeutically effective amount of one or more agents used for treating atheromatous plaques is administered as part of the disclosed combination therapy.

U.S. patent application Ser. No. 16/186,495 (“the '495 application”) discloses a combination therapy for the removal of atheromatous plaques from coronary arteries. See also Ahmad, A. “A Cure for Coronary Artery Disease,” J. Diabetes Metab. 2017, 8(10), 769, doi: 10.4172/2155-6156.1000769. The pathology of atheromatous plaques in cerebral arteries is the same as the pathology of atheromatous plaques in coronary arteries. Thus a similar treatment regimen should be effective for removal of atheromatous plaques from the cerebrovascular region. The '495 application discloses the use of a combination of one or more agents that reduce insulin resistance, one or more anti-inflammatory agents, and one or more PCSK9 inhibitors for the removal of atheromatous plaques from coronary arteries. While the '495 application discloses a specific treatment regimen using specific agents to reduce insulin resistance and inflammation and to inhibit PCSK9, the general principles underlying the combination therapy disclosed in the '495 application may be applied to the use of other agents with similar properties for the treatment of atheromatous plaques.

In some embodiments, a therapeutically effective amount of use of a combination of one or more agents that reduce insulin resistance, one or more anti-inflammatory agents, and one or more PCSK9 inhibitors for the removal of atheromatous plaques in the cerebrovascular region is administered as part of the disclosed combination therapy.

In some embodiments, one or more the anti-inflammatory agents are selected from the group consisting of one or more statins, eicosapentaenoic acid or a derivative thereof, and montelukast.

Statins are HMG-CoA (HMOA) reductase inhibitors used to lower cholesterol and LDL levels and, in some cases, also increase HDL levels. Notably, statins are also anti-inflammatory agents. Blake, G. J., et al. “Are Statins Anti-Inflammatory?” Curr. Control. Trials Cardiovasc. Med. 2000, 1, 161-65.

In some embodiments, the one or more anti-inflammatory agents comprise a statin. In some embodiments, the statin is selected from the group consisting of atorvastatin, simvastatin, rosuvastatin, and pravastatin. In some preferred embodiments, the statin is atorvastatin.

Omega-3 fatty acids, in particular eicosapentaenoic acid (EPA) and its derivatives, have anti-inflammatory properties and also reduce triglyceride levels in the blood. See, e.g., Ito, M. K. “A Comparative Overview of Prescription Omega-3 Fatty Acid Products,” P&T, 2015, 40(12), 826-57. The ethyl ester of EPA, icosapent ethyl, is particularly effective for the reduction of triglyceride levels in the blood. Id.

In some embodiments, the one or more anti-inflammatory agents comprise icosapent ethyl.

Montelukast is a leukotriene receptor antagonist and has anti-inflammatory properties. See, e.g., Jones, T. R., et al. “Pharmacology of Montelukast Sodium (Singulair), A Potent and Selective Leukotriene D4 Receptor Antagonist,” Can. J. Physiol. Pharmacol. 1995, 73(2), 191-201.

In some embodiments, the one or more anti-inflammatory agents comprise montelukast.

In some embodiments, the one or more anti-inflammatory agents comprise atorvastatin, icosapent ethyl, and montelukast.

Alirocumab, a human monoclonal antibody, is a PCSK9 inhibitor that lowers LDL cholesterol levels. See Am. Coll. Cardiol. Press Release, “Alirocumab Reduces Cardiovascular Events after Acute Coronary Syndrome,” Mar. 10, 2018. Alirocumab is being studied for use in removal of atheromatous plaques in intracranial arteries. See de Havanon, A. “PCSK9 Inhibition in Patients With Symptomatic Intracranial Atherosclerosis,” Early Phase 1 Clinical Trial, study available online at clinicaltrials.gov/ct2/show/NCT03507374.

Evolocumab, a human monoclonal antibody, is a PCSK9 inhibitor used to treat hyperlipidemia. Combination therapy using evolocumab at its maximum dose along with statins may significantly lower LDL levels. See, e.g., Nicholls, S. J., et al. “Effect of Evolocumab on Progression of Coronary Artery Disease in Statin-Treated Patients: The GLAGOV Randomized Clinical Trial,” J. Am. Med. Assoc. 2016, 16, 2373-84.

In some embodiments, the PCSK9 inhibitor is alirocumab. In some alternate embodiments, the PCSK9 inhibitor is evolocumab.

### Treatment of Chronic Inflammation of the Brain

Certain cytokines are generated in the brain of a patient with Alzheimer's disease that are not generated in the brains of healthy patients. See, e.g., Zheng, C., et al. “The Dual Roles of Cytokines in Alzheimer's Disease: Update on Interleukins, TNF-α, TGF-β and IFN-γ,” Transl. Neurodegener. 2016, 5, 7, doi: 10.1186/s40035-016-0054-4. These cytokines cause inflammation, and it is believed that the production of these cytokines is related to the formation of amyloid β plaques (Aβ) and neurofibrillary tangles (NFT). See, e.g., Id.; Domingues, C., et al. “Impact of Cytokines and Chemokines on Alzheimer's Disease Neuropathological Hallmarks,” Curr. Alzheimer Res. 2017, 14(8), 870-82, doi: 10.2174/1567205014666170317113606. Neuro-inflammation is related to cytokine production, including chemokines and TNF-α. See, e.g., Fuster-Matanzo, A., et al., supra. Neuro-inflammation may lead to neurogenesis, but may also lead to the destruction of neurons. See, e.g., Fuster-Matanzo, A., et al., supra. Astrocytes and microglia are abundant near the neurons and amyloid β plaques. See, e.g., Zheng, C., et al., supra; Zhang, F., et al. “Neuroinflammation in Alzheimer's Disease,” Neuropsychiatr. Dis. Treat. 2015, 11, 243-56, doi: 10.2147/NDT.S75546.

Neuro-inflammation in patients with Alzheimer's disease may be treated with one or more anti-inflammatory agents. In some embodiments, the one or more anti-inflammatory agents may comprise one or more agents selected from the group consisting of one or more statins, eicosapentaenoic acid or a derivative thereof, and montelukast.

In some embodiments, a therapeutically effective amount of one or more anti-inflammatory agents is administered as part of the disclosed combination therapy.

In some embodiments, the one or more anti-inflammatory agents comprise a statin. In some embodiments, the statin is selected from the group consisting of atorvastatin, simvastatin, rosuvastatin, and pravastatin. In some preferred embodiments, the statin is atorvastatin.

In some embodiments, the one or more anti-inflammatory agents comprise icosapent ethyl.

In some embodiments, the one or more anti-inflammatory agents comprise montelukast.

In some embodiments, the one or more anti-inflammatory agents comprise atorvastatin, icosapent ethyl, and montelukast.

### Neurogenesis

Neurogenesis occurs in the sub-ventricular region of lateral ventricles and the sub-granular region of dentate gyrus of the hippocampus. See Fujimaki, S., et al. “Diabetes and Stem Cell Function,” BioMed Res. Int. 2015, doi: 10.1155/2015/592915. Insulin and IGF signaling play an important role in controlling neural stem cell differentiation, determining the fate of neural stem cells, and the migration and survival of neurons. See Id. Thus, by reducing insulin resistance and also administering one or more agents that promote neurogenesis it is possible to remedy the neuron loss that is characteristic of Alzheimer's disease.

The anti-depressant paroxetine is known to increase hippocampal neurogenesis. See, e.g., Peng, Z. W., et al. “Paroxetine Up-Regulates Neurogenesis in Hippocampus-Derived Neural Stem Cell from Fetal Rats,” Mol. Cell. Biochem. 2013, 375(1-2), 105-13, doi: 10.1007/s11010-012-1533-2.

Omega-3 fatty acids, including eicosapentaenoic acid (EPA), are also known to enhance neurogenesis. See, e.g., Crupi, R., et al. “n-3 Fatty Acids: Role in Neurogenesis and Neuroplasticity,” Curr. Med. Chem. 2013, 20(24), 2953-63.

In some embodiments, a therapeutically effective amount of use of a combination of one or more agents that reduce insulin resistance and one or more agents that promote neurogenesis is administered as part of the disclosed combination therapy.

In some embodiments, the one or more agents that promote neurogenesis are selected from the group consisting of paroxetine and eicosapentaenoic acid or a derivative thereof.

In some embodiments, the one or more agents that promote neurogenesis comprise paroxetine.

In some embodiments, the one or more agents that promote neurogenesis comprise eicosapentaenoic acid or a derivative thereof. In some embodiments, the one or more agents that promote neurogenesis comprises icosapent ethyl.

In some embodiments, the one or more agents that promote neurogenesis comprise paroxetine and eicosapentaenoic acid or a derivative thereof. In some embodiments, the one or more agents that promote neurogenesis comprise paroxetine and icosapent ethyl.

It should be understood that pharmaceutically acceptable salts, prodrugs, or derivatives of one or more of the agents described herein may be used instead of the agents described, without loss of efficacy.

### EXAMPLE

The disclosed treatment regimen offers significant advantages over current treatments for patients with Alzheimer's disease. The example described below is provided as a specific illustration. It should be understood, however, that the invention is not limited to the specific details set forth in the example, such as the specific agents selected to achieve desired outcomes and the dosages used.

A 67 year old patient suffering from significant memory loss and vision loss was evaluated for treatment. The patient suffered a gradual decline in both short-term and long-term memory over at least three years. The patient also complained about vision loss over a period of about six years. The patient scored 11 out of 30 on a mental status exam, indicating dementia or other cognitive impairment.

A clinical psychologist determined that the patient was oriented to person, but poorly oriented to place and time. He did not know his home address, his telephone number, or the city of his residence, although he did know the state of his residence. The patient acknowledged having anger problems. The patent denied having anxiety, mania, or depression but acknowledged occasional suicidal ideation. He denied having any current intent to harm himself. The patient's wife reported that the patient would sometimes sit and rock back-and-forth. She also reported that the patient would sometimes hit his head when upset. There was no evidence that the patient suffered from delusions or hallucinations, and there was no evidence of agnosia.

The patient previously had coronary artery bypass surgery and also suffered from type 2 diabetes mellitus for multiple decades. The patient suffered a myocardial infarction in 2006 and also had a history of multiple transient ischemic attacks (TIAs). The patient also suffered from hepatitis C, but was not previously treated. It was determined that the patient was only able to perceive light in both eyes. The patient's loss of vision was due to glaucoma and diabetic retinopathy. The patient also had urinary incontinence that was controlled with oxybutynin.

The patient's comprehensive metabolic panel testing was normal. Tests for syphilis and HIV were negative, and the patient's blood mercury level was normal.

A magnetic resonance image (MRI) scan of the patient's brain showed extensive damage, while a magnetic resonance angiogram (MRA) scan was normal. The MRI scan showed chronic encephalomalacia in the right temporal and occipital lobes. There were chronic ischemic changes in the pons, basal ganglia, and sub-cortical white matter.

Neither an amyloid PET scan of the brain nor an examination of CSF were conducted.

The patient was taking the following medications before changes in his treatment were made:
1. insulin—Lantus 20 units twice per day, Novolog with meals.
2. memantine (Namenda XR)—21 mg per day
3. donepezil—5 mg per day
4. oxybutynin—5 mg per day
5. clopidogrel bisulfate (Plavix)—75 mg per day
6. alprazolam—0.25 mg three times per day as needed
7. fluoxetine—20 mg per day
   The following medications were added to the patient's treatment to treat and cure the patient's Alzheimer's disease:
8. chromium picolinate—800 μg per day
9. magnesium oxide—400 mg twice per day
10. coenzyme Q10-100 mg three times per day
11. alirocumab—300 mg subcutaneous once per month
12. atorvastatin—40 mg per day
13. icosapent ethyl (Vascepa)—1 g twice per day
14. montelukast (Singulair)—10 mg per day
15. lysine—1 g three times per day
   The patient's other medications were continued.

The treatment protocol involved the following for treatment of all the major etiological factors for Alzheimer's disease:
(1) the use of lysine to treat the herpes simplex virus infection;
(2) the use of insulin to control diabetes mellitus and chromium picolinate, magnesium oxide, and coenzyme Q10 to further reduce insulin resistance;
(3) reducing insulin resistance to treat amyloid plaques and neurofibrillary tangles;
(4) treating the cerebral atheromatous plaques to treat the arteriosclerosis of the cerebral arteries, and treating amyloid angiopathy by reducing insulin resistance;
(5) reducing insulin resistance to treat the endothelial dysfunction of blood vessels and lack of angiogenesis;
(6) the use of atorvastatin, icosapent ethyl, and montelukast to treat the chronic inflammation of the brain; and
(7) reducing insulin resistance and the use of paroxetine and icosapent ethyl to treat the extensive loss of neurons and impaired neurogenesis.
   The patient was examined by a different clinical psychologist after the treatment as described above was administered for seven months, and found that the patient no longer suffered from dementia. The patient's vision also improved.

Prior treatment with medications such as memantine and donepezil was unsuccessful because the treatment did not address all the etiological factors of Alzheimer's disease. The treatment regimen described cured the patient of Alzheimer's disease because it addressed all the etiological factors.

The previous description of the disclosed embodiments is provided to enable any person skilled in the art to make or use the invention disclosed herein. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

All references cited herein are expressly incorporated by reference.

What is claimed is:

1. A method of treating Alzheimer's disease in a patient comprising administering a therapeutically effective amount of:
a. one or more agents used for treatment of the herpes simplex virus;
b. one or more agents used for reducing insulin resistance;
c. one or more anti-inflammatory agents;
d. one or more agents for treatment of atheromatous plaques; and
e. one or more agents for treating impaired neurogenesis.

2. The method of claim 1, wherein the one or more agents used for treatment of the herpes simplex virus comprise lysine.

3. The method of claim 1, wherein the one or more agents used reducing insulin resistance are selected from the group consisting of insulin, chromium picolinate, magnesium oxide, coenzyme Q10, carvedilol, and metformin.

4. The method of claim 2, wherein the one or more agents used reducing insulin resistance are selected from the group consisting of insulin, chromium picolinate, magnesium oxide, coenzyme Q10, carvedilol, and metformin.

5. The method of claim 4, wherein the one or more agents used reducing insulin resistance are selected from the group consisting of insulin, chromium picolinate, magnesium oxide, and coenzyme Q10.

6. The method of claim 1, wherein the one or more anti-inflammatory agents are selected from the group consisting of one or more statins, eicosapentaenoic acid or a derivative thereof, and montelukast.

7. The method of claim 6, wherein the one or more anti-inflammatory agents are selected from the group consisting of atorvastatin, eicosapentaenoic acid or a derivative thereof, and montelukast.

8. The method of claim 2, wherein the one or more anti-inflammatory agents are selected from the group consisting of one or more statins, eicosapentaenoic acid or a derivative thereof, and montelukast.

9. The method of claim 2, wherein the one or more anti-inflammatory agents are selected from the group consisting of atorvastatin, eicosapentaenoic acid or a derivative thereof, and montelukast.

10. The method of claim 4, wherein the one or more anti-inflammatory agents are selected from the group consisting of one or more statins, eicosapentaenoic acid or a derivative thereof, and montelukast.

11. The method of claim 4, wherein the one or more anti-inflammatory agents are selected from the group consisting of atorvastatin, eicosapentaenoic acid or a derivative thereof, and montelukast.

12. The method of claim 5, wherein the one or more anti-inflammatory agents are selected from the group consisting of one or more statins, eicosapentaenoic acid or a derivative thereof, and montelukast.

13. The method of claim 5, wherein the one or more anti-inflammatory agents are selected from the group consisting of atorvastatin, eicosapentaenoic acid or a derivative thereof, and montelukast.

14. The method of claim 1, wherein the one or more agents for treatment of atheromatous plaques comprise one or more agents that reduce insulin resistance, one or more anti-inflammatory agents, and one or more PCSK9 inhibitors.

15. The method of claim 13, wherein the one or more agents for treatment of atheromatous plaques comprise one or more agents that reduce insulin resistance, one or more anti-inflammatory agents, and one or more PCSK9 inhibitors.

16. The method of claim 15, wherein the one or more PCSK9 inhibitors is selected from the group consisting of alirocumab and evolocumab.

17. The method of claim 1, wherein the one or more agents for treating impaired neurogenesis are selected from the group consisting of paroxetine and eicosapentaenoic acid or a derivative thereof.

18. The method of claim 15, wherein the one or more agents for treating impaired neurogenesis are selected from the group consisting of paroxetine and eicosapentaenoic acid or a derivative thereof.

19. The method of claim 16, wherein the one or more agents for treating impaired neurogenesis are selected from the group consisting of paroxetine and eicosapentaenoic acid or a derivative thereof.

20. A method of treating Alzheimer's disease in a patient comprising administering a therapeutically effective amount of:
a. lysine;
b. insulin;
c. chromium picolinate;
d. magnesium oxide;
e. coenzyme Q10;
f. atorvastatin;
g. icosapent ethyl;
h. montelukast; and
i. alirocumab.
